(19) United States
(12) Patent Application Publication
(10) Pub. No.: US 2013/0210147 A1
(43) Pub. Date: Aug. 15, 2013

(54) Bioactive amino acid sequence and use therefrom

(71) Applicants: Laurent Jeannin, Brussels (BE); Roland Callens, Tielt (BE); Wafa Moussa, Nice (FR)

(72) Inventors: Laurent Jeannin, Brussels (BE); Roland Callens, Tielt (BE); Wafa Moussa, Nice (FR)

(73) Assignee: SOLVAY SA, Brussels (BE)

(21) Appl. No.: 13/877,915

(22) PCT Filed: Oct. 6, 2011
(86) PCT No.: PCT/EP11/67481

§ 371 Date: Apr. 5, 2013

(30) Foreign Application Priority Data
Oct. 8, 2010 (EP) 10187069.9
Oct. 8, 2010 (EP) 10187070.7

(51) Int. Cl. C12N 5/071

(52) U.S. Cl. CPC C12N 5/0625
USPC 435/377; 435/375; 435/397

(57) ABSTRACT

Use of the amino acid sequence Har-Gly-Asp (hRGD) as a bioactive sequence in functional peptides to promote cell adhesion, cell growth, and/or cell differentiation, and in the preparation of hydrogels, preferably hydrogels for cell culture. A hydrogel comprising the hRGD sequence, especially a hydrogel wherein the hRGD sequence is part of the hydrogel scaffold.

The present application claims the benefit of the European applications No. 10187070.7 and 10187069.9, both filed on Oct. 8, 2010, herein incorporated by reference.

### DESCRIPTION

The present invention relates to a bioactive amino acid sequence and its use in a functional peptide, in particular to promote cell adhesion, cell growth and/or cell differentiation.

Bioactive or biologically active peptide (or amino acid) sequences are known in the art. Bioactive sequences may be derived from any of a diverse range of naturally occurring proteins and peptides including ECM components, cell adhesion molecules, cell surface receptors, growth factors, cytokines, chemokines, etc. For example, the -RGD-sequence is a prototypic cell recognition sequence found in fibronectin and well known to be recognized by integrins and to mediate cell attachment. The integrin-mediated cell attachment influences and regulates cell migration, growth, differentiation, and apoptosis.

Despite the availability of bioactive amino acid sequences promoting cell adhesion, cell growth and/or cell differentiation, such as -RGD-, there is a continuous need for new sequences, showing improved properties, for instance in view of specific cell types or in specific conditions, in particular in the fields of drug delivery or cell and tissue culture.

Accordingly, the purpose of the present invention, is to provide a bioactive amino acid sequence that has particularly advantageous properties to promote cell adhesion, cell growth and/or cell differentiation.

The present invention therefore relates to the use of the amino acid sequence Har-Gly-Asp (hRGD) as a bioactive sequence in a functional peptide to promote cell adhesion, cell growth and/or cell differentiation.

The inventors have indeed surprisingly found that this amino acid sequence exhibits mainly improved cell adhesion, and cell growth. In particular, the present amino acid sequence shows a longer half-life time, in particular via a stronger resistance towards enzymatic degradation. The present amino acid sequence also shows higher affinity towards specific cell lines.

By “bioactive sequence” or “biologically active sequence” is intended an amino acid sequence which has a specific biological function, here the promotion of cell adhesion (or cell attachment), cell growth and/or cell differentiation (or induction of a cellular phenotype). An example cell differentiation is the transformation of pluri or omni potent cells, for example stem cells, into dedicated cell types, such as bone cells, muscle cells, insulin secreting cells etc.

As used herein, the term “amino acid” (Xaa) is intended to denote any compound comprising at least one NR₁R₂ group, preferably NH₂ group, and at least one carboxyl group. The amino acids of this invention can be natural amino acids or non-natural amino acids, naturally occurring or synthetic. The natural amino acids, with exception of glycine, contain a chiral carbon atom. Unless otherwise specifically indicated, the compounds containing natural amino acids with the L-configuration are preferred. The aminoacids can be selected from, for example β-alanine (βAla), γ-aminobutyric acid (GABA), 5-aminovaleric acid, glycine (Gly or G), phenylglycine, arginine (Arg or R), homoarginine (Har or hR), alanine (Ala or A), valine (Val or V), norvaline, leucine (Leu or L), norleucine (Nle), isoleucine (Ile or I), serine (Ser or S), isoserine, homoserine (Hse), threonine (Thr or T), allothreonine, methionine (Met or M), ethionine, glutamic acid (Glu or E), aspartic acid (Asp or D), asparagine (Asn or N), cysteine (Cys or C), cystine, phenylalanine, tyrosine (Tyr or Y), tryptophan (Trp or W), lysine (Lys or K), hydroxylysine (Hyl), histidine (His or H), ornithine (Orn), glutamine (Gln or Q), citrulline, proline (Pro or P), and 4-hydroxyproline (Hyp or O).

As used herein, the term “peptide” comprises peptides and peptide analogous. Peptide analogous comprise natural amino acids and non-natural amino acids. They can also comprise modifications such as glycosylations. All amino acids can be either the L- or D-isomer. The peptides or peptide analogues can also comprise amino acid mimetics that function in a manner similar to the naturally occurring amino acids. The peptides may also be formed from amino acids analogues that have modified R groups or modified peptide backbones. Peptide analogues usually include at least one bond in the peptide sequence which is different from an amide bond, such as urethane, urea, ester or thioester bond. Peptides or peptide analogues according to the present invention can be linear, cyclic or branched and are preferably linear.

By “functional peptide moiety” is intended a peptide moiety comprising a bioactive sequence, thus a peptide moiety exhibiting a biological activity.

In the present invention, the amino acid sequence Har-Gly-Asp (hRGD) can represent in itself the bioactive sequence of a functional peptide moiety, promoting cell adhesion, cell growth and/or cell differentiation.

In a particular embodiment, the amino acid sequence Har-Gly-Asp (hRGD) can also be part of a longer bioactive sequence of a functional peptide moiety, promoting cell adhesion, cell growth and/or cell differentiation.

In a first aspect of this particular embodiment, the hRGD sequence may comprise additional amino acids covalently bound to its N-terminus (NH₂). The bioactive sequence may for instance be selected from (Xaa)ₙ-hRGD sequences wherein Xaa is any natural or unnatural amino acid and n is 1 to 10. The n Xaa amino acids may be the same or different. Suitable examples of such sequences are GhRGD, YhRGD, YGhRGD, GGGGhRGD, βAla-hRGD, GABA-hRGD and 6-aminovalericamide-hRGD.

In a second aspect of this particular embodiment, the hRGD sequence may comprise additional amino acids covalently bound to its C-terminus (COOH). The bioactive sequence may for example be selected from hRGD-(Xaa)ₘ sequences wherein Xaa is any natural or unnatural amino acid and m is 1 to 10. The m Xaa amino acids may be the same or different. Suitable examples of such sequences are hRGDS, hRGDY, hRGDF, hRGDK, hRGDV, hRGDT hRGDWP, hRGDYK, hRGDFK, hRGDSP, hRGDSPK, hRGDSY, hRGDNP, hRGDTP, and hRGDSP, in particular hRGDWP.

In a third aspect of this particular embodiment, the first and second aspects as described above may be combined, the hRGD sequence comprising additional amino acids covalently bound to both its N- and C-terminus, i.e. (Xaa)ₙ-hRGD-(Xaa)ₘ where Xaa is any natural or unnatural amino acid, n is 1 to 10, and m is 1 to 10. Such sequences may for instance be selected from the group consisting of GhRGDS, GhRGDY, GhRGDF, YGhRGD, GhRGDSY, GhRGDSP, GhRGDSPK, YhRGDS, GhRGDTP, GhRGDSPK, GhRGDSP, GhRGDK, GGGGhRGDS, GhRGDNP, and combinations thereof; in particular GhRGDS, GhRGDSY.

The present invention therefore also relates to the use of a bioactive sequence in a functional peptide to promote cell adhesion, cell growth and/or cell differentiation, wherein the bioactive sequence is selected from the group consisting of GhRGD, YhRGD, YGhRGD, GGGGhRGD, βAla-hRGD, and GABA-hRGD, 6-aminovalericamide-hRGD, hRGDS, hRGDY, hRGDF, hRGDK, hRGDV, hRGDT, hRGDWP, hRGDFK, hRGDYK, hRGDSP, hRGDSPK, hRGDSY, hRGDNP, hRGDTP, and hRGDSP, GhRGDS, GhRGDY, GhRGDF, GhRGDSY, GhRGDSP, GhRGDSPK, YhRGDS, GhRGDTP, GhRGDSPK, GhRGDSP, GhRGDK, GGGGhRGDS, GhRGDNP, and combinations thereof; in particular hRGDS, GhRGDS, GhRGDSY, βAla-hRGD, GABA-hRGD, 6-aminovalericamide-hRGD, and hRGDWP; more particularly βAla-hRGD, GABA-hRGD, 6-aminovalericamide-hRGD, and hRGDWP; most preferably hRGDWP.

In a further particular embodiment, the hRGD sequence, optionally comprising additional amino acids on its N- and/or C-terminus, can also be linked to mercaptopropionic acid (Mpr) on its N-terminus. Such sequences can read Mpr-(Xaa)ₙ-hRGD-(Xaa)ₘ wherein Xaa is any natural or unnatural amino acid selected independently from one another, n is 0 to 10, and m is 0 to 10. In a still further embodiment, two mercaptopropionic acid moieties may be covalently bound together, in particular via a disulfur bond. Such sequences typically read (Xaa)ₘ-DGhR-(Xaa)ₙ-Mpr-Mpr-(Xaa)_{n′}-hRGD-(Xaa)_{m′} wherein (Xaa) is any natural or unnatural amino acid selected independently from one another, n and n′ range independently from 0 to 10, and m and m′ range independently from 0 to 10.

In the present invention, the functional peptide moiety can correspond to the bioactive sequence comprising the hRGD sequence. The functional peptide moiety can also comprise additional amino acids, further to the bioactive sequence comprising the hRGD sequence.

According to a particularly preferred embodiment, the functional peptide moiety comprises at least one or more hRGD sequences, such as two, three, four, five, six, seven, eight, nine or ten hRGD sequences, preferably at least one or more hRGDWP sequences, such as two, three, four, five, six, seven, eight, nine or ten hRGDWP sequences. In this particularly preferred embodiment, the functional peptide moiety may comprise additional amino acids before, after or between bioactive sequences as defined above.

hRGDWP sequences and their derivatives provide the advantage of mimicking cell adhesion proteins in the extracellular matrix and subsequently can bind integrin proteins on the cell surface.

In a first aspect of the present invention, the functional peptide moiety comprising the bioactive amino acid sequence Har-Gly-Asp (hRGD) is covalently bound to a self-assembling peptide moiety.

By “self-assembling peptide” is intended a peptide able to self-assemble, i.e. a peptide defining a domain that folds into a specifically defined conformation in contrast with non-self-assembling peptide domains having many random conformations. Self-assembling amino acid sequences are known in the art and, according to the present invention, peptide sequences capable of assembling into a β-sheet, a coiled coil α-helix structure, a peptide triple helix structure, or combinations thereof are preferred.

A peptide moiety that is capable of self-assembly into a coiled coil structure is, for example, a peptide amino acid sequence providing an α-helical coiled coil structure. This is a tertiary structure which depends on the amphiphilic pattern of the peptides primary sequence. The peptide moiety of this embodiment comprises a variety of hydrophobic and polar residues, and is usually composed of at least 10 amino acids. For example, the helix peptide moiety is designed to have all the polar residues on one face of the helix and all the hydrophobic residues on the other side of the helix. This helix can form part of two or more helix chains and form a coiled coil structure. The helices are associated together through hydrophobic interaction and form a coiled coil. The sequence of the peptide moiety can for example be a leucine zipper sequence.

Peptide moieties capable of self-assembling into a β-sheet provide β-sheet stabilized by inter-molecular hydrogen bonding perpendicular to the peptide chain. The self-assembling occurs through hydrogen bond interactions between beta strands. The beta strand is a stretch of polypeptide chain with a backbone in an almost fully extended conformation. The β-sheet structure can be formed either from parallel or anti-parallel β-strands. An example of a β-sheet according to this embodiment is a peptide moiety that is able to self-assemble in an amyloid-like structure. Peptide moieties capable of self-assembling into a β-sheet comprise typically at least 5 or 6 amino acids.

According to a preferred embodiment of this first aspect of the present invention, the peptide moiety that is able to self-assemble, can form a hydrogel when the peptide is provided in suitable conditions. Hydrogels are three-dimensional networks of hydrophilic compounds, usually polymers, which have the ability to imbibe a large quantity of water and biological fluids. The network may be formed through either chemical crosslinking (covalent, ionic) or physical crosslinking (entanglements, crystallites, hydrogen bonds). Typically, hydrogels are three-dimensional structures capable of comprising at least 20 wt % water in relation to the weight of the gel. Absorption of water by a hydrogel gel results in a significant increase of its dimensions, i.e. a significant swelling.

According to another preferred embodiment of this first aspect of the present invention, the peptide moiety that is able to self-assemble into a β-sheet is an octapeptide moiety comprising alternating hydrophobic and charged amino acids. Hydrophobic amino acids are often selected from the group consisting of Phenylalanine (Phe or F), Tryptophan (Trp or W), Tyrosine (Tyr or Y), Isoleucine (Ile or I), Alanine (Ala or A), Leucine (Leu or L), Valine (Val or V), and Norleucine (Nle); in particular from Phenylalanine (Phe or F), Tryptophan (Trp or W), Tyrosine (Tyr or Y), Isoleucine (Ile or I), and Norleucine (Nle). Charged amino acids are usually selected from the group consisting of Arginine (Arg or R), Aspartic acid (Asp or D), Glutamic acid (Glu or E), Lysine (Lys or K), and Histidine (His or H); particularly from Arginine (Arg or R), Aspartic acid (Asp or D), Glutamic acid (Glu or E), and Lysine (Lys or K).

The octapeptide moiety might for instance be selected from the group consisting of FEFKFEFK, FEFEFKFK, FDFKFDFK, FDFDFKFK, FEFRFEFR, FEFEFRFR, YDYKYDYK, YDYDYKYK, YEYRYEYR, YEYKYEYK, YEYEYKYK, WEWKWEWK, WEWEWKWK, WDWKWDWK, WDWDWKWK. Most preferably the amino sequences are FEFKFEFK or FEFEFKFK.

In a second aspect of the present invention, the functional peptide moiety comprising the bioactive amino acid sequence Har-Gly-Asp (hRGD) is covalently bound to a polymer, in particular to a biocompatible polymer, more particularly to a thermo-responsive polymer.

By “biocompatible polymer” is intended a polymer which is compatible with living organisms. Suitable examples are polyesters like polylactic acid (PLA), polyglycolic acid (PGA), poly(lactic-co-glycolic acid) (PLGA), polyhydroxyalkanoates like poly(hydroxyl butyrate-co-valerate) (PHBV), polycaprolactones, polyacrylamides like polyhydroxyacrylamide (pHPMA), polyanhydrides, polyimide, polyanhydride-co-imide, and polysaccharides like chitosan.

By “thermo-responsive polymer” is intended a polymer which undergoes a physical change, such as conformational change, when exposed to external thermal stimuli such as an increase, or decrease, in temperature. The ability of thermo-responsive polymers to undergo physical changes in response to thermal stimuli classifies these polymers in the art in the category of smart materials.

Contrary to the behavior of most polymers in aqueous solutions, thermal-responsive polymers become less soluble, or more hydrophobic, in water at elevated temperatures. The temperature providing the above phase transition from soluble to insoluble is designated in the art as the lower critical solution temperature (LCST), generally determined in deionized water at a neutral pH. An especially suitable thermo-responsive polymer is poly(N-isopropylacrylamide) or PNIPAAm which LCST has been determined to be approximately 32° C. Other especially suitable thermo-responsive polymers are PNIPAAm copolymers. PNIPAAm copolymers are copolymers comprising NIPAAm and at least one other monomer, the other monomer being selected from hydrophilic or hydrophobic monomers. Suitable hydrophilic monomers are for instance as N-methacryloyl-tris(hydroxymethyl)methylamide, hydroxyethyl acrylamide, hydroxypropyl methacrylamide (HPMA), N-acrylamido-1-deoxysorbitol, hydroxyl-ethylmethacrylate, hydroxypropylactrylate, hydroxyphenyl methacrylate, 2-hydroxypropyl acrylate, 4-hydroxybutylmethactrylate, 2-methacryloxyethyl glucoside, poly(ethyleneglycol)monomethyl ether monomethacrylate, vinyl-4-hydroxybutyl ether, and derivatives thereof; poly(ethylene glycol) containing monomers being especially suitable, as well as hydroxypropylmethacrylamide (HPMA). Suitable hydrophobic monomers are derived from acrylamide monomers in which the amine nitrogen of the amide group is substituted with one or more alkyl residues, for example N-isopropylacrylamide, N,N-dimethylacrylamide, N,N-diethyl(meth)acrylamide, N-methyl methacrylamide, N-ethylmethacrylamide, N-propylacrylamide, N-butylacrylamide, N-octyl(meth)acrylamide, N-dodecylmethacrylamide, N-octadecylacrylamide, propyl(meth)acrylate, decyl(meth)acrylate, stearyl(meth)acrylate, octyl-triphenylmethylacrylamide, butyl-triphenylmethylacrylamide, octadedcyl-triphenylmethylacrylamide, phenyl-triphenylmethylacrylamide, benzyl-triphenylmethylacrylamide, and derivatives thereof.

Thermo-responsive polymers can be used for the preparation of hydrogels and more particularly for the preparation of smart hydrogels, i.e. environmental sensitive hydrogels. These smart hydrogels can undergo a reversible volume change in response to environmental stimuli such as pressure, pH, temperature or ionic strength making them especially suitable to be used in biomedical and pharmaceutical fields, in particular in the field of cell and tissue culturing.

In this second specific aspect of the present invention, the functional peptide moiety comprising the bioactive amino acid sequence Har-Gly-Asp (hRGD) is preferably covalently bound to the polymer through a linkage, preferably selected from the group consisting of thioether, amino, amido, ester and ether linkage. Such linkage can be directly, i.e. a covalent bond between the atoms of the polymer and the atoms of the functional peptide, or indirectly, i.e., through a linking group. Suitable examples of linking groups are among others linear or branched alkanes, especially polymethylene group comprising 1 to 10 carbon atoms. Other examples of linking groups are for instance polyether groups, such as polyethylene glycol (PEG).

In a third aspect of the present invention, the functional peptide moiety comprising the hRGD sequence is covalently bound to both a self-assembling peptide and a polymer, in particular to both a self-assembling peptide and a biocompatible polymer, more particularly to both a self-assembling peptide and a thermo-responsive polymer.

In a further embodiment the present invention also relates to the use of the amino acid sequence Har-Gly-Asp (hRGD) as a bioactive sequence in a functional peptide to promote cell adhesion, cell growth and/or cell differentiation, in the preparation of hydrogels, preferably hydrogels for cell culture.

The present invention also relates to hydrogels comprising the hRGD sequence, especially hydrogels wherein the hRGD sequence is part of the hydrogel scaffold. By “hydrogel scaffold” is meant the material leading to the hydrogel structure, i.e. a physical entity comprising a polymer or a self-assembling peptide that will give rise to the hydrogel structure. In said hydrogels, the hRGD sequence as defined above preferably promotes cell adhesion, cell growth and/or cell differentiation.

In further preferred embodiments, the hydrogels of the present invention further comprise a self-assembling peptide moiety and/or a polymer as defined above. In a still further preferred embodiment, the hRGD sequence is covalently bound to at least one of the self-assembling peptide moiety or polymer as defined above, optionally via a linkage and/or an additional peptide sequence. The present invention therefore also relates to a hydrogel comprising the hRGD sequence and a self-assembling peptide moiety and/or a polymer, in particular a self-assembling peptide moiety and/or a biocompatible polymer, more particularly a self-assembling peptide moiety and/or a thermo-responsive polymer.

The present hydrogels are especially suitable for cell and tissue culture, providing, for example, improved cell adhesion and cell growth. The present hydrogel can be used for example for culturing cells, preferably fibroblast cells, chondrocyte cells or stem cells, or for tissue engineering.

The present hydrogels may be used in 2- or 3-dimensional cell culture systems.

Should the disclosure of any patents, patent applications, and publications which are incorporated herein by reference conflict with the description of the present application to the extent that it might render a term unclear, the present description shall take precedence.

### DESCRIPTION OF THE FIGURES

FIG. 1: optical micrographs of hydrogels based on octapeptide, hRGD and RGD modified octapeptides after 1, 3 and 7 days.

FIG. 2: fluorescence optical micrographs of hydrogels based on octapeptide, hRGD and RGD modified octapeptides after 1, 3 and 11 days.

FIG. 3: optical micrographs of hydrogels based on octapeptide, hRGD and RGD modified octapeptides after 1 day.

FIG. 4: fluorescence optical micrographs of hydrogels based on octapeptide, hRGD and RGD modified octapeptides after 1 and 3 days.

FIG. 5: cell number after 0, 1, 2, 3, 5, 7, 10 and 14 days in hydrogels based on octapeptide, RGD and hRGD modified octapeptides.

The present invention is further illustrated below without limiting the scope thereto.

### EXAMPLES

### Example 1

In the following, (h)RGD means that RGD, hRGD or a mixture thereof can be used.

### 1.1 Synthesis of the Protected Octapeptide

Octapeptide Phe-Glu-Phe-Lys-Phe-Glu-Phe-Lys (FEFKFEFK) can be synthesized as disclosed in A. Maslovskis et al., Macromol. Symp., 296, 248-253 (2010), on a ChemTech ACT 90 peptide synthesizer using N-methyl-2-pyrrolidone (NMP) as solvent, and standard solid phase peptide protocols.

Octapeptide can be also synthesized in a liquid phase approach (strategies Z/Boc/OtBu or Fmoc/Boc/OtBu). In this particular case, side protection groups remain on the sequence even during the deprotection of the protecting group on N-terminal position, thus leading to Z-Phe-Glu(OtBu)-Phe-Lys(Boc)-Phe-Glu(OtBu)-Phe-Lys(Boc)-OH.

### 1.2 Esterification of the Protected Octapeptide

This esterification step was performed according the literature: P. Jouin et al., J. Org. Chem., 54, 3, 617-626, 1989. 50 g of Z-Phe-Glu(OtBu)-Phe-Lys(Boc)-Phe-Glu(OtBu)-Phe-Lys(Boc)-OH (32 mmol) and 5.3 g of cesium carbonate were introduced in 500 ml of N,N-dimethylformamide (DMF). 5.5 ml of iodoethane (EtI) were added, and the solution was heated at 48° C. for 2 hours. After filtration of the salts and partial evaporation of DMF, the concentrate was poured into 500 ml of KHSO₄ 2.5%, filtrated, washed with water and finally with 500 ml warm ethanol. After drying under vacuum (45° C.), 47 g of a solid, corresponding to Z-Phe-Glu(OtBu)-Phe-Lys(Boc)-Phe-Glu(OtBu)-Phe-Lys(Boc)-OEt were obtained. Yield=84%.

### 1.3 Hydrogenolysis of the Protected Octapeptide Ethyl Ester

21.6 g of Z-Phe-Glu(OtBu)-Phe-Lys(Boc)-Phe-Glu(OtBu)-Phe-Lys(Boc)-OEt (13.6 mmol) were dissolved in 215 ml of N,N-dimethylformamide (DMA). After flushing the solution several times with nitrogen, 14.5 g of Pd/Si (2% weight) were added. Hydrogenolysis was initiated by the introduction of hydrogen. After 2 hours of reaction, the suspension was passed through a 0.45 μm filter and Pd/Si was washed by DMA. The gathered filtrates, corresponding to H-Phe-Glu(OtBu)-Phe-Lys(Boc)-Phe-Glu(OtBu)-Phe-Lys(Boc)-OEt, were used without further purification in the next steps. The yield was quantitative.

### Example 2

### Synthesis of Protected Mpr-hRGDWP

7.3 g of S-trityl (or triphenylmethyl) mercaptopropionyl homoarginyl glycine (Mpr(Trityl)-Har-Gly-OH) (12.0 mmol) were dissolved in 75 ml of N,N-dimethylformamide (DMF) containing 1 ml of pyridine. Once the solution cooled at −10±5° C., 1.6 g of pivaloyl chloride (PivCl) were added. After 5-10 min of activation, 6.3 g of O-t-butyl aspargyl tryptophanyl proline (Asp(OtBu)-Trp-Pro) (12.7 mmol) solubilized in 10 ml of DMF containing 6.1 g of N-trimethylsilylacetamide (TMA) were added. The reaction mixture was then brought back to room temperature.

After HPLC control of the completion of the reaction, 16.4 ml of water were added. After partial concentration, the concentrate diluted by 35 ml of methanol was poured into 155 ml of NaHCO₃ aqueous 2.5%. The precipitate was washed several times with water, and dried under vacuum. 10.6 g of an off-white product corresponding to Mpr(Trt)-Har-Gly-Asp(OtBu)-Trp-Pro-OH were obtained. Yield=87%.

### Example 3

### 3.1 Coupling of the Protected Mpr-hRGDWP and Octapeptide

13.9 g of Mpr(Trt)-Har-Gly-Asp(OtBu)-Trp-Pro-OH (13.2 mmol), 2.5 g of p-toluenesulfonic acid, and 6.2 g of hydroxybenzotriazole (HOBt) were added to a solution of H-Phe-Glu(OtBu)-Phe-Lys(Boc)-Phe-Glu(OtBu)-Phe-Lys(Boc)-OEt (12 mmol) in N,N-dimethylformamide (DMA). Once a solution obtained, 2.7 g of 1-ethyl-3-β-dimethylaminopropyl)carbodiimide hydrochloride (EDC) were added at room temperature. After stirring at least 8 hours, the reaction mixture was poured into 300 ml of KHSO₄ 2.5% aqueous. After filtration, the precipitate was washed three times by ethanol (270 ml), and dried under vacuum at 45° C. 26.3 g of an off-white solid, corresponding to the sequence Mpr(Trt)-Har-Gly-Asp(OtBu)-Trp-Pro-Phe-Glu(OtBu)-Phe-Lys(Boc)-Phe-Glu(OtBu)-Phe-Lys(Boc)-OEt, were obtained. Yield=72%.

### 3.2 Final Deprotection of the Protected Mpr-hRGDWP-Octapeptide

5 g of Mpr(Trt)-Har-Gly-Asp(OtBu)-Trp-Pro-Phe-Glu(OtBu)-Phe-Lys(Boc)-Phe-Glu(OtBu)-Phe-Lys(Boc)-OEt (1.9 mmol) were then introduced into a solution containing 100 ml of TFA, 100 ml of CH₂Cl₂, 9 ml of tri-isopropylsilane ((iPr)₃SiH), 2.2 ml of water and 2.2 ml of EtOH. After about 1 hour of reaction, the reaction mixture was poured into 200 ml of cold isopropyl ether (IPE). After filtration and washing several times by IPE, the peptide was dried under vacuum (45° C.). 3 g of off-white peptide were obtained, corresponding to Mpr-Har-Gly-Asp-Trp-Pro-Phe-Glu-Phe-Lys-Phe-Glu-Phe-Lys-OEt. Yield=88%.

In order to increase the purity of this sequence, preparative HPLC following by lyophilization can be used.

### Example 4

### Synthesis of Protected Mpr-RGDWP

### 4.1 Synthesis of Z-Arg-Gly-Asp(OtBu)-Trp-Pro-OH

3.4 g of Z-Arg-Gly-OH (10.0 mmol) were dissolved in 80 ml of a mixture dichloromethane (CH₂Cl₂)/N,N-dimethylacetamide (DMA) (1/1) containing 0.86 ml of pyridine. Once the solution cooled at −10±5° C., 1.3 g of pivaloyl chloride (PivCl) were added. After 5-10 min of activation, 5.21 g of Asp(OtBu)-Trp-Pro (10.6 mmol) solubilized in 0.3 ml of CH₂Cl₂ containing 5.29 g of N-trimethylsilylacetamide (TMA) were added. The reaction mixture was then brought back to room temperature.

After HPLC control of the completion of the reaction, 10 ml of water were added. After partial concentration, the concentrate diluted by 30 ml of methanol was poured into 140 ml of NaHCO₃ aqueous 2.5%. The precipitate was washed several times with water.

9.1 g of the resulting wet product, corresponding to Z-Arg-Gly-Asp(OtBu)-Trp-Pro-OH, were added to about 200 ml of a boiling mixture of acetonitrile/water/methanol (79/20/1 v/v). After solubilisation of the solid, the solution was progressively cooled down. During this cooling down step, 340 ml of NaHCO₃ aqueous 2.5% were poured. After apparition of a white solid, the suspension was further stirred at 0° C. for at least 10 h. After filtration and several washings by 100 ml of acetonitrile/water (1/2), the peptide was dried under vacuum (45° C.). 6 g of off-white peptide were obtained, corresponding to Z-Arg-Gly-Asp(OtBu)-Trp-Pro-OH (5% water content). Yield=70%.

### 4.2 Synthesis of H-Arg-Gly-Asp(OtBu)-Trp-Pro-OH

20.0 g of Z-Arg-Gly-Asp(OtBu)-Trp-Pro-OH (22.2 mmol) were dissolved in 240 ml of a mixture methanol/water (95/5) containing 1.85 ml of HCl 37% aqueous. After flushing the solution several times with nitrogen, 23.6 g of Pd/Si (2% weight) were added. Hydrogenolysis was initiated by the introduction of hydrogen. After 2 hours of reaction, the suspension was passed through a 0.45 μm filter and Pd/Si was washed by a mixture methanol/water (95/5). The gathered filtrates were concentrated under vacuum, and water was further replaced through azeotropic concentration by acetonitrile. The precipitate was filtrated, washed and dried under vacuum. 13.1 g of off-white powder, corresponding to H-Arg-Gly-Asp(OtBu)-Trp-Pro-OH.HCl, were obtained (1.8% water content). Yield=85%.

### 4.3 Synthesis of Mpr(Trt)-Arg-Gly-Asp(OtBu)-Trp-Pro-OH

8.08 g of H-Arg-Gly-Asp(OtBu)-Trp-Pro-OH.HCl (11 mmol) were added at 45° C. to 150 ml of a mixture water/dioxane (1/2) at a pH ranging from 8.0 and 8.5 (KHCO₃ buffer). 5 g of Mpr(Trt)OSu (10.4 mmol), divided into 5 equivalent fractions, were added at regular interval to the above solution. After control of the completion of the reaction by HPLC, the reaction mixture was poured into 400 ml of water and the dioxane fraction was evaporated under vacuum. After filtration of the suspension, the wet solid was treated according the protocol described in example 4.1, to lead to Mpr(Trt)-Arg-Gly-Asp(OtBu)-Trp-Pro-OH (protected Mpr-RGDWP). Yield=74%.

### Example 5

### 5.1 Coupling of the Protected Mpr-RGDWP and Octapeptide

The Mpr(Trt)-Arg-Gly-Asp(OtBu)-Trp-Pro-OH was then coupled to H-Phe-Glu(OtBu)-Phe-Lys(Boc)-Phe-Glu(OtBu)-Phe-Lys(Boc)-OEt according the protocol used in Example 3.2. Yield=74%.

### 5.2 Final Deprotection of the Protected Mpr-RGDWP-Octapeptide

The protected Mpr-RGDWP-octapeptide was then deprotected according the protocol used in Example 3.2 for the deprotection of the protected Mpr-hRGDWP-octapeptide. Yield=87%.

In order to increase the purity of this sequence, preparative HPLC following by lyophilization can be used.

### Example 6

### Hydrogel Preparation

### 6.2 Protocol I

### 6.2.1 2D Cell Culture

16.3 mg of octapeptide and 7.3 mg of hRGD-octapeptide or 6.6 mg of RGD-octapeptide (80/20 octapeptide/(h)RGD-octapeptide molar ratio) were dissolved in 1 ml of distilled water at 90° C. for 3 hours. On cooling, the samples were transferred in the cell culture well plate and incubated at 37° C. for 12 hours. The samples were then washed for 10 minutes with cell culture medium (DMEM Gibco, Invitogen) by changing the medium over the gel 8 times. The samples were placed back in the incubator at 37° C. overnight and washes were repeated the next day. The samples were placed again overnight in the incubator and the next day cells were seeded on the surface of the gels.

### 6.2.2 3D Cell Culture

16.3 mg of octapeptide and 7.3 mg of hRGD-octapeptide or 6.6 mg pf RGD-octapeptide (80/20 octapeptide/(h)RGD-octopeptide molar ratio) were dissolved in 1 ml of a mixture of distilled water and 1× Dulbecco's Phosphate Buffered Saline (70/30 ratio) at 90° C. for 3 hours. On cooling, 500 μl of sample were placed in each cell culture well plates with 40 μl of 0.5M NaOH solution and stirred. Then 100 μl of cells suspended in medium were added to the wells and stirred in. 100 μl of cell culture medium were then added on top of the gels in the wells and the well plate were placed in the incubator.

### 6.1 Protocol II

125 μl of NaOH 2N were added to 5.1 ml of Dulbecco's Modified Eagle Medium (DMEM). The solution was vigorously shaken and rapidly added to 715 μl of a dimethylsulfoxide (DMSO) solution containing the a mixture octapeptide/(h)RGDWP-octapeptide (80/20 w/w) (concentration 200 mg peptide/ml DMSO). The immediate formed gel was shaken for 3 min. The obtained gel was washed on a 5 μm membrane four times by 15 ml Dulbecco's Modified Eagle Medium (DMEM). The washed gel was directly used for 2D cell culture or then transferred into a vial, slightly diluted by DMEM (5-10% of the gel volume), and shaken for few minutes. The suspended gel could then be transferred into culture flasks and be inoculated with cells for 3D cell culture.

### Example 7

### Cell Culture

In the following tests, three hydrogels were compared: a hydrogel based on self-assembling octapeptide FEFKFEFK, a hydrogel based on a RGD modified octapeptide (Mpr-RGDWP-FEFKFEFK), and a hydrogel based on a hRGD modified octapeptide (Mpr-hRGDWP-FEFKFEFK).

The cell morphology and cell attachment were observed using optical microscopy.

### 7.1 2D Tests

### 7.1.1 Cell Morphology

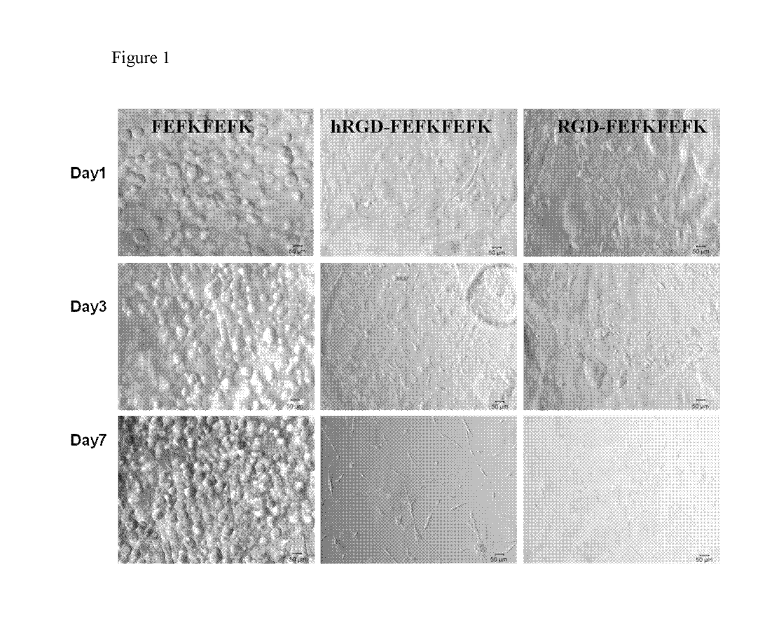

The cell attachment properties of Human Dermal Fibroblasts (HDF) on the gel surfaces were investigated. FIG. 1 shows optical microscopy pictures of hydrogels based on octapeptide, hRGD and RGD modified octapeptides, taken after 1, 3 and 7 days.

It can be seen from these pictures that cells seeded on FEFKFEFK hydrogel surfaces demonstrated a rounded morphology after days 1, 3 and 7 in culture. An extensive cell elongation was observed on hRGD modified hydrogel surfaces during the 7 days culture period whereas cells adhered to RGD modified hydrogel surfaces revealed relatively less stretched cell morphology between day 1 and day 3. From the optical micrographs, it can be seen that cell elongation on hRGDWP-FEFKFEFK hydrogels is superior compared to cell elongation on RGDWP-FEFKFEFK hydrogels.

### 7.1.2 Cell Viability

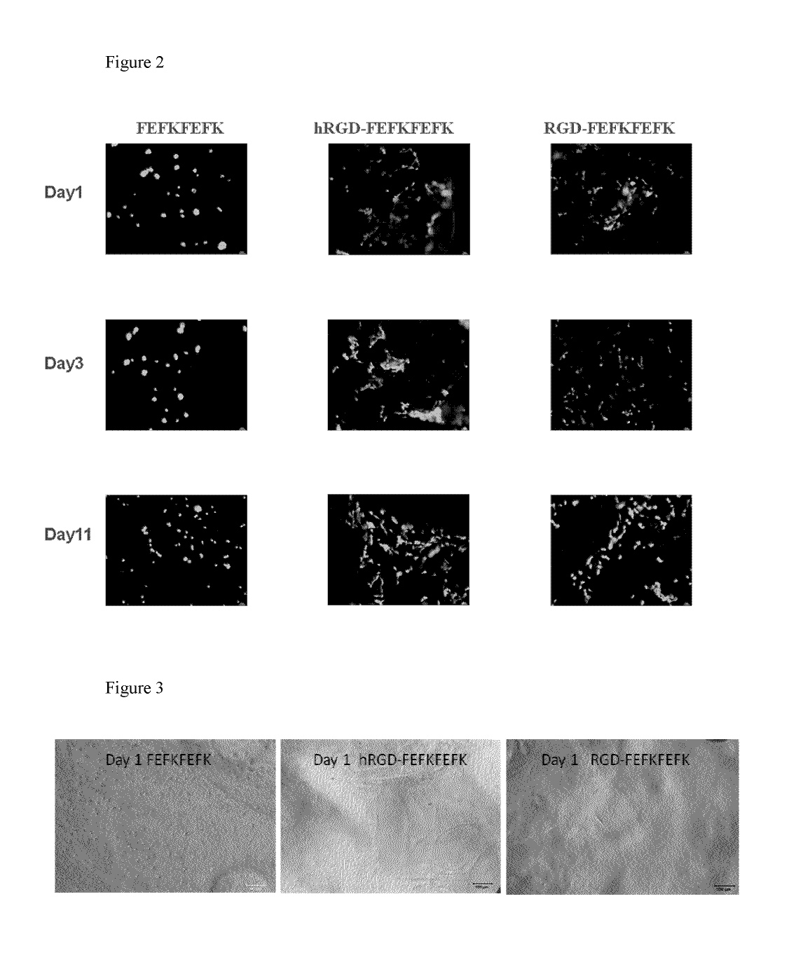

Cell viability of HDF's on the hydrogel surfaces was visually analyzed by optical microscopy using live-dead staining. Calcein AM (non-fluorescent) was converted to calcein (intensely fluorescent) via intracellular esterase activity achieved through enzymatic reaction. This intensely fluorescence calcein was retained within living cells producing a green fluorescence. The damaged membrane of the dead cells allowed EthD-1 solution to bind to nucleic acids thereby producing a red fluorescence in dead cells. FIG. 2 shows fluorescence optical microscopy pictures of hydrogels based on octapeptide, hRGD and RGD modified octapeptides, taken after 1, 3 and 11 days. The green fluorescence corresponding to living cells appears in light grey in the pictures. No red fluorescence was observed.

The fluorescence micrographs show that majority of the cells on FEFKFEFK hydrogel have a rounded morphology up to day 7 in culture. Very few dead cells were recorded. In contrast, images revealed that after day 1 and day 3 in culture, the HDF's seeded on hRGD modified hydrogels showed a highly stretched morphology compared to the ones seeded on RGD modified gels. These results correlated well with the optical microscopy images presented in example 7.1.1. Medium change after alternating days provided sufficient nutrients to facilitate cell growth; consequently very few dead cells were detected.

### 7.2 3D tests

As in 2D tests, the behavior of Human Dermal Fibroblasts (HDF) was analyzed.

### 7.2.1 Cell Morphology

As in example 7.1.1, the cell morphology of Human Dermal Fibroblasts (HDF) on the gel surfaces was investigated. FIG. 3 shows optical microscopy pictures of hydrogels based on octapeptide, hRGD and RGD modified octapeptides, taken after 1 day.

It was evident that cells encapsulated in FEFKFEFK hydrogels demonstrated a rounded morphology after day 1 in culture. Extensive cell elongation was observed within the hRGD modified hydrogels whereas cells embedded in RGD modified hydrogels revealed relatively less stretched cell morphology at day 1. Significant difference in cell morphology was observed between non-modified and modified hydrogels. From the optical micrographs it can be seen that cell elongation within hRGDWP-FEFKFEFK hydrogels is superior to cell elongation within RGDWP-FEFKFEFK hydrogels.

### 7.2.2 Cell Viability

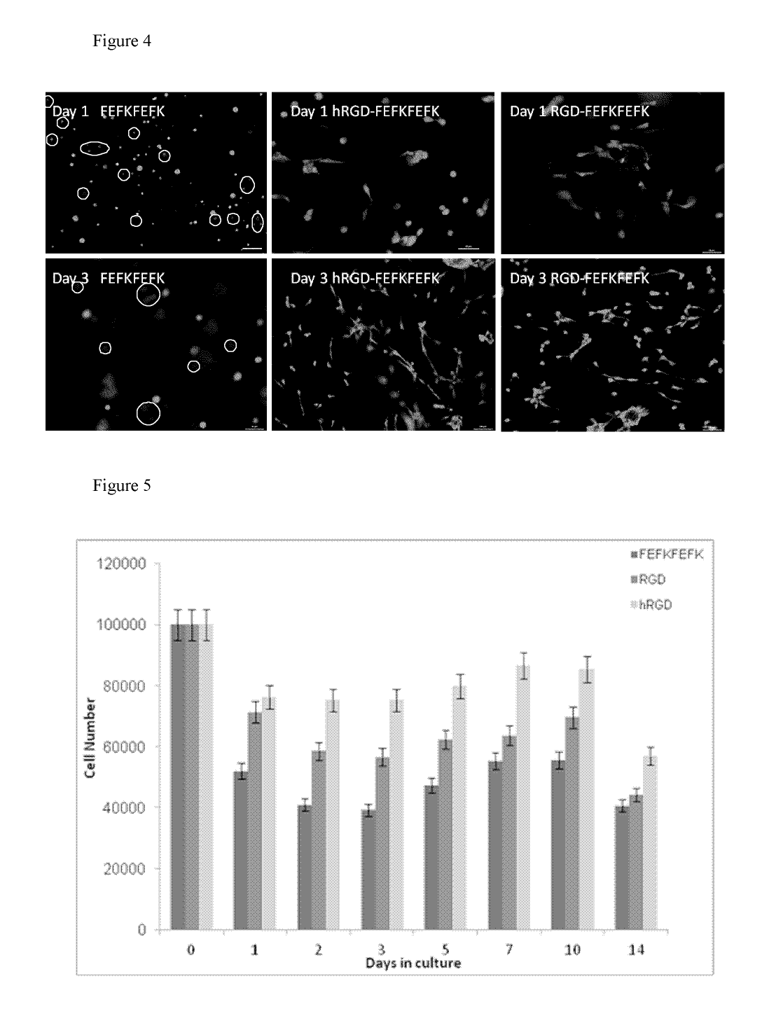

As in example 7.1.2, cell viability of HDF's encapsulated within the gels was visually analysed using live-dead staining. FIG. 4 shows fluorescence optical microscopy pictures of hydrogels based on octapeptide, hRGD and RGD modified octapeptides, taken after 1 and 3 days. The green fluorescence, corresponding to living cells, appears in light grey in the pictures. Red fluorescence, corresponding to dead cells, is present of the pictures corresponding to the octapeptide (circled spots).

The fluorescence micrographs show that majority of the cells encapsulated within the FEFKFEFK hydrogel demonstrated a rounded morphology up to day 3 in culture. Dead cells were observed at day 1. At day 3 the cell viability improved and therefore, few dead cells were detected. In contrast, it can be seen from the images that between day 1 and day 3, HDF's embedded within the hRGD modified hydrogels demonstrated a highly stretched morphology compared to the HDF's embedded within the RGD modified hydrogels.

### 7.2.3 Cell Proliferation

A cell count method using a haemocytometer was performed to quantify the cell proliferation of HDF's in 3D culture system. FIG. 5 shows the cell number after respectively 0, 1, 2, 3, 5, 7, 10 and 14 days in hydrogels based on octapeptide, RGD and hRGD modified octapeptides.

Highest cell numbers and percentage of cells alive were observed in the hRGD modified hydrogel. Lowest cell numbers were observed in the unmodified FEFKFEFK system. Cell numbers in RGD modified hydrogel steadily increased with time but the percentage alive and the degree of cell proliferation varied. The large difference between hRGD and RGD modified hydrogels is thought to be partly due to the cell response to the difference in stiffness between the gels. The rheology data gathered show distinct differences in the strength and β-sheet content of the three hydrogels (see Table 1).

TABLE 1

| | Rheology | |
|---|---|---|
| FEFKFEFK hydrogel | ~1000 | Pa |
| RGD modified hydrogel | ~2000-3000 | Pa |
| hRGD modified hydrogel | ~10000 | Pa |

1. A method for promoting cell adhesion, cell growth, cell differentiation, or combinations thereof, comprising using an amino acid sequence Har-Gly-Asp (hRGD) as a bioactive sequence in a functional peptide moiety.

2. The method according to claim 1, wherein the functional moiety comprises at least one sequence selected from the group consisting of GhRGD, YhRGD, YGhRGD, GGGGhRGD, βAla-hRGD, GABA-hRGD, 6-aminovalericamide-hRGD, hRGDS, hRGDY, hRGDF, hRGDK, hRGDV, hRGDT, hRGDWP, hRGDFK, hRGDYK, hRGDSP, hRGDSPK, hRGDSY, hRGDNP, hRGDTP, hRGDSP, GhRGDS, GhRGDY, GhRGDF, GhRGDSY, GhRGDSP, GhRGDSPK, YhRGDS, GhRGDTP, GhRGDSPK, GhRGDSP, GhRGDK, GGGGhRGDS, GhRGDNP, and combinations thereof.

3. The method according to claim 1, wherein the functional peptide moiety is covalently bound to a self-assembling peptide moiety.

4. The method according to claim 3, wherein the self-assembling peptide moiety is able to self-assemble in a β-sheet, a coiled coil α-helix structure, or a peptide triple helix structure.

5. The method according to claim 1, wherein the functional peptide moiety is covalently bound to a biocompatible polymer.

6. The method according to claim 5, wherein the functional peptide moiety is bound to the biocompatible polymer through a linkage selected from the group consisting of a thioether linkage, an amino linkage, an amido linkage, an ester linkage, and an ether linkage.

8. The method according to claim 3, being used for the preparation of hydrogels.

9. A hydrogel comprising a Har-Gly-Asp (hRGD) sequence.

10. The hydrogel according to claim 9, wherein the hRGD sequence is part of the hydrogel scaffold.

11. The hydrogel according to claim 10, wherein the hRGD sequence promotes cell adhesion, cell growth, cell differentiation, or combinations thereof.

12. The hydrogel according to claim 10, wherein the hydrogel further comprises a self-assembling peptide moiety, a polymer, or both a self-assembling peptide moiety and a polymer.

13. The hydrogel according to claim 12, wherein the hRGD sequence is covalently bound to at least one of the peptide moiety or polymer.

14. The hydrogel according to claim 12, wherein the hRGD sequence is covalently bound to at least one of the peptide moiety or polymer via an additional peptide sequence.

15. The hydrogel according to claim 12, wherein the hRGD sequence is covalently bound to at least one of the peptide moiety or polymer via a linkage.

16. The method according to claim 2, wherein the functional moiety comprises at least one sequence selected from the group consisting of hRGDS, GhRGDS, GhRGDSY, βAla-hRGD, GABA-hRGD, 6-aminovalericamide-hRGD, and hRGDWP.

17. The method according to claim 2, wherein the functional moiety comprises at least one sequence selected from the group consisting of βAla-hRGD, GABA-hRGD, 6-aminovalericamide-hRGD, and hRGDWP.

18. The method according to claim 2, wherein the functional peptide moiety is covalently bound to a self-assembling peptide moiety.

19. The method according to claim 5, wherein the biocompatible polymer is a thermo-responsive polymer


SEQUENCE LISTING (60 sequences)

SEQ ID NO: 1 - protein, 4 aa, Artificial
  Synthetic peptide
GXGD

SEQ ID NO: 2 - protein, 4 aa, Artificial
  Synthetic peptide
YXGD

SEQ ID NO: 3 - protein, 5 aa, Artificial
  Synthetic peptide
YGXGD

SEQ ID NO: 4 - protein, 7 aa, Artificial
  Synthetic peptide
GGGGXGD

SEQ ID NO: 5 - protein, 4 aa, Artificial
  Synthetic peptide
XXGD

SEQ ID NO: 6 - protein, 4 aa, Artificial
  Synthetic peptide
XXGD

SEQ ID NO: 7 - protein, 4 aa, Artificial
  Synthetic peptide
XXGD

SEQ ID NO: 8 - protein, 4 aa, Artificial Sequence
  Synthetic peptide
XGDS

SEQ ID NO: 9 - protein, 4 aa, Artificial
  Synthetic peptide
XGDY

SEQ ID NO: 10 - protein, 4 aa, Artificial
  Synthetic peptide
XGDF

SEQ ID NO: 11 - protein, 4 aa, Artificial
  Synthetic peptide
XGDK

SEQ ID NO: 12 - protein, 4 aa, Artificial
  Synthetic peptide
XGDV

SEQ ID NO: 13 - protein, 4 aa, Artificial
  Synthetic peptide
XGDT

SEQ ID NO: 14 - protein, 5 aa, Artificial
  Synthetic peptide
XGDWP

SEQ ID NO: 15 - protein, 5 aa, Artificial
  Synthetic peptide
XGDYK

SEQ ID NO: 16 - protein, 5 aa, Artificial
  Synthetic peptide
XGDFK

SEQ ID NO: 17 - protein, 5 aa, Artificial
  Synthetic peptide
XGDSP

SEQ ID NO: 18 - protein, 6 aa, Artificial
  Synthetic peptide
XGDSPK

SEQ ID NO: 19 - protein, 5 aa, Artificial
  Synthetic peptide
XGDSY

SEQ ID NO: 20 - protein, 5 aa, Artificial
  Synthetic peptide
XGDNP

SEQ ID NO: 21 - protein, 5 aa, Artificial
  Synthetic peptide
XGDTP

SEQ ID NO: 22 - protein, 5 aa, Artificial
  Synthetic peptide
XGDSP

SEQ ID NO: 23 - protein, 5 aa, Artificial
  Synthetic peptide
GXGDS

SEQ ID NO: 24 - protein, 5 aa, Artificial
  Synthetic peptide
GXGDY

SEQ ID NO: 25 - protein, 5 aa, Artificial
  Synthetic peptide
GXGDF

SEQ ID NO: 26 - protein, 6 aa, Artificial
  Synthetic peptide
GXGDSY

SEQ ID NO: 27 - protein, 6 aa, Artificial
  Synthetic peptide
GXGDSP

SEQ ID NO: 28 - protein, 7 aa, Artificial
  Synthetic peptide
GXGDSPK

SEQ ID NO: 29 - protein, 5 aa, Artificial
  Synthetic peptide
YXGDS

SEQ ID NO: 30 - protein, 6 aa, Artificial
  Synthetic peptide
GXGDTP

SEQ ID NO: 31 - protein, 7 aa, Artificial
  Synthetic peptide
GXGDSPK

SEQ ID NO: 32 - protein, 5 aa, Artificial
  Synthetic peptide
GXGDK

SEQ ID NO: 33 - protein, 8 aa, Artificial
  Synthetic peptide
GGGGXGDS

SEQ ID NO: 34 - protein, 6 aa, Artificial
  Synthetic peptide
GXGDNP

SEQ ID NO: 35 - protein, 8 aa, Artificial Sequence
  Synthetic peptide
FEFKFEFK

SEQ ID NO: 36 - protein, 8 aa, Artificial Sequence
  Synthetic peptide
FEFEFKFK

SEQ ID NO: 37 - protein, 8 aa, Artificial
  Synthetic peptide
FDFKFDFK

SEQ ID NO: 38 - protein, 8 aa, Artificial
  Synthetic peptide
FDFDFKFK

SEQ ID NO: 39 - protein, 8 aa, Artificial
  Synthetic peptide
FEFRFEFR

SEQ ID NO: 40 - protein, 8 aa, Artificial
  Synthetic peptide
FEFEFRFR

SEQ ID NO: 41 - protein, 8 aa, Artificial
  Synthetic peptide
YDYKYDYK

SEQ ID NO: 42 - protein, 8 aa, Artificial
  Synthetic peptide
YDYDYKYK

SEQ ID NO: 43 - protein, 8 aa, Artificial
  Synthetic peptide
YEYRYEYR

SEQ ID NO: 44 - protein, 8 aa, Artificial
  Synthetic peptide
YEYKYEYK

SEQ ID NO: 45 - protein, 8 aa, Artificial
  Synthetic peptide
YEYEYKYK

SEQ ID NO: 46 - protein, 8 aa, Artificial
  Synthetic peptide
WEWKWEWK

SEQ ID NO: 47 - protein, 8 aa, Artificial
  Synthetic peptide
WEWEWKWK

SEQ ID NO: 48 - protein, 8 aa, Artificial
  Synthetic peptide
WDWKWDWK

SEQ ID NO: 49 - protein, 8 aa, Artificial
  Synthetic peptide
WDWDWKWK

SEQ ID NO: 50 - protein, 8 aa, Artificial Sequence
  Synthetic peptide
FEFKFEFK

SEQ ID NO: 51 - protein, 8 aa, Artificial Sequence
  Synthetic peptide
FEFKFEFK

SEQ ID NO: 52 - protein, 8 aa, Artificial Sequence
  Synthetic peptide
FEFKFEFK

SEQ ID NO: 53 - protein, 5 aa, Artificial
  Synthetic peptide
XGDWP

SEQ ID NO: 54 - protein, 13 aa, Artificial Sequence
  Synthetic peptide
XGDWPFEFKFEFK

SEQ ID NO: 55 - protein, 13 aa, Artificial Sequence
  Synthetic peptide
XGDWPFEFKFEFK

SEQ ID NO: 56 - protein, 5 aa, Artificial
  Synthetic peptide
RGDWP

SEQ ID NO: 57 - protein, 5 aa, Artificial
  Synthetic peptide
RGDWP

SEQ ID NO: 58 - protein, 13 aa, Artificial Sequence
  Synthetic peptide
RGDWPFEFKFEFK

SEQ ID NO: 59 - protein, 13 aa, Artificial Sequence
  Synthetic peptide
XGDWPFEFKFEFK

SEQ ID NO: 60 - protein, 13 aa, Artificial Sequence
  Synthetic peptide
RGDWPFEFKFEFK